(19) United States
(12) Patent Application Publication
(10) Pub. No.: US 2023/0309983 A1
(43) Pub. Date: Oct. 5, 2023

(54) INCIDENTAL DUROTOMY CLOSURE SYSTEM AND METHOD

(71) Applicant: THE BOARD OF TRUSTEES OF THE UNIVERSITY OF ILLINOIS, URBANA, IL (US)

(72) Inventors: Ankit Mehta, Chicago, IL (US); Clayton Rosinski, Iowa City, IA (US)

(21) Appl. No.: 18/131,140

(22) Filed: Apr. 5, 2023

Related U.S. Application Data
(60) Provisional application No. 63/327,408, filed on Apr. 5, 2022.

(51) Int. Cl. A61B 17/04

(52) U.S. Cl. CPC A61B 17/04; A61B 2017/0464

(57) ABSTRACT

The disclosure provides an example implant, surgical systems, and methods for closing a tissue opening. The implant includes a barbed suture having a first end and a second end. A first disc statically coupled to the first end of the barbed suture at a center of the first disc. And a second disc having a through-hole arranged in a center of the second disc, where the second disc is moveably coupled to the barbed suture via the through-hole.

### CROSS-REFERENCE TO RELATED APPLICATION

This application is a U.S. Non-Provisional application that claims priority to U.S. Provisional Patent Application No. 63/327,408, filed Apr. 5, 2022, which is hereby incorporated by reference in its entirety.

### BACKGROUND

In surgeries of the bony portion of the spinal column that are often performed for back pain or nerve compression, a common complication is incidental durotomy. Incidental durotomy occurs when a tear is accidentally created in the dura during the surgical procedure. The dura mater is the fibrous covering and outer limit of the thecal sac, which includes the spinal cord, the cerebrospinal fluid (“CSF”) that bathes and protects the spinal cord, and the meninges that surround the spinal cord and CSF. The tear must be closed intraoperatively in order to prevent the formation of a CSF leak that occurs when the CSF is able to exit the thecal sac.

A CSF leak can lead to many delayed complications such as headache, prolonged wound healing, and infection, amongst others, and may necessitate reoperation to close the durotomy if it does not spontaneously resolve. This comes with a financial burden, as the cost to both the hospital and patient are increased when a durotomy occurs.

Currently, the gold standard of incidental durotomy repair is primary closure, where the tear is closed via suturing. Great care must be taken when closing a durotomy with suture to ensure that the spinal cord is not injured by the needle, that the closure does not cause kinking of the thecal sac, which could result in spinal cord compression, and that the durotomy is entirely closed so CSF does not leak. Thus, the repair procedure can be lengthy and complicated.

Further, primary closure with suture is typically augmented with a secondary closure procedure, which often involves placement of a patch over the site of durotomy and application of a sealant with a goal to reinforce the closure. In cases where the durotomy cannot be closed via suture due to a location not being amenable to suturing or when the dural defect is complex, only secondary closure is attempted with hopes that the dural patch and sealant will be sufficient. Ultimately, the presence of a durotomy adds time to the operation, mental fatigue for the surgeon, the chance for postoperative complications and possibly reoperation.

Although incidental durotomy is being used as the example, this principle of closing an opening in a barrier tissue is not isolated to spine surgery, but a common practice of many other surgical fields. For instance, in laparoscopic surgery, where surgical instruments and a camera are passed through small incisions made in the abdomen and advanced through the peritoneum, i.e., the connective tissue which forms the outer limit of the abdominal cavity, these so-called port sites must be closed at the end of the procedure to prevent abdominal organs from herniating out of the abdomen. Additional examples of where tissue openings must be surgically closed include, but are not limited to, operations that take place within a joint, within the spinal cord, on intrauterine fetuses, within blood vessels, hollow organ perforation, or burr hole closure.

### SUMMARY

In a first aspect, an example implant for closing a tissue opening is disclosed. The implant includes (a) a barbed suture having a first end and a second end, (b) a first disc statically coupled to the first end of the barbed suture at a center of the first disc; and (c) a second disc having a through-hole arranged in a center of the second disc, where the second disc is moveably coupled to the barbed suture via the through-hole.

In a second aspect, an example surgical system for closing a tissue opening is disclosed. The surgical system includes (a) the implant according to the first aspect of the disclosure, and (b) a delivery device having a lumen with a first end having a delivery opening, a second end, an inner diameter, and an outer diameter, where the implant is disposed in the lumen of the delivery device such that the first disc is arranged adjacent to the delivery opening of the lumen and the second disc is arranged in the lumen between the first disc and the second end of the lumen, and where the second end of the lumen is coupled to a force-imparting guide.

In a third aspect, an example method of closing a tissue opening during a surgical procedure using the surgical system according to the second aspect of the disclosure is provided. The method includes (a) inserting the first end of the delivery device into the tissue opening, (b) advancing the first disc of the implant out of the delivery opening of the lumen and through the tissue opening, the first disc thereby expanding into a deployment condition adjacent a first side of the tissue opening, (c) retracting the first end of the delivery device from the tissue opening such that the barbed suture extends through the tissue opening, (d) advancing the second disc of the implant out of the delivery opening of the lumen, the second disc thereby expanding into a deployment condition adjacent a second side of the tissue opening; and (e) advancing the second disc toward the tissue opening and the first disc until the second disc is in facial contact with the second side of the tissue opening thereby sealing the tissue opening between the first disc and the second disc.

The features, functions, and advantages that have been discussed can be achieved independently in various examples or may be combined in yet other examples further details of which can be seen with reference to the following description and drawings.

### BRIEF DESCRIPTION OF THE DRAWINGS

FIG. 1 depicts a side view of an implant in a deployment condition, according to an example implementation;

FIG. 2 depicts a side view of a surgical system that includes the implant in the delivery condition, according to an example implementation;

FIG. 3 depicts a side view of a surgical system with a first end of delivery device place in a tissue opening, according to an example implementation;

FIG. 4 depicts a side view of a surgical system with a first disc of an implant deployed through the tissue opening and into a target location, according to an example implementation;

FIG. 5 depicts a side view of a surgical system with the first disc and a second disc of the implant deployed on opposing sides of the tissue opening, according to an example implementation;

FIG. 6 depicts a side detail view of the implant in a deployed condition with the second disc advanced toward the first disc on opposing sides of the tissue opening, according to an example implementation;

FIG. 7 depicts a side view of the implant in a deployed condition with the first disc and the second disc of the implant forming a seal around the tissue opening with the barbed suture pulled taut, according to an example implementation;

FIG. 8 depicts a side view of the implant in a deployed condition with the first disc and the second disc of the implant forming a seal around the tissue opening with the excess barbed suture removed from the second disc, according to an example implementation;

FIG. 9 depicts a side view of the implant in a deployed condition with the first disc and the second disc of the implant forming a seal around the tissue opening in the thecal sac with the excess barbed suture removed from the second disc, according to an example implementation; and

FIG. 10 shows a flowchart of a method for closing a tissue opening with an implant, according to an example implementation.

The drawings are for the purpose of illustrating examples, but it is understood that the disclosure is not limited to the arrangements and instrumentalities shown in the drawings.

### DETAILED DESCRIPTION

Implants, surgical systems, and methods constructed in accordance with the principles herein are designed to quickly close or initiate closure of durotomies or other tissue openings during a surgical procedure such as, but not limited to, laparoscopic port sites, the amnion for fetal procedures, the joint capsule in arthroscopic procedures, burr holes in neurosurgical procedures, or any hollow organ perforation.

The specific benefits of the disclosed implant, in the context of incidental durotomy, among others, are reduced durotomy repair time, reduced surgeon fatigue, improved durotomy closure, and synergy with current secondary repair devices. In patients undergoing lumbar spine surgery, incidental durotomy repair has been shown to prolong the surgical case by an average of 55.6 minutes (249.8±100.3 minutes vs 194.2±95.4 minutes). This is due to the time taken to carefully suture the durotomy closed, then test the closure intraoperatively.

Implants and surgical systems of the present improve incidental durotomy repair that can now be closed in a process that takes just seconds to minutes once it is in the field. Additionally, implants and surgical systems herein offer the ability to primarily close durotomies which may be inaccessible for closure by suturing. For example, durotomies which are on the side of the thecal sac near the boney elements of the spine are often difficult to suture due to limitations of space. With the implants and surgical systems herein, as long as a delivery device can be introduced to the durotomy, the implant can be delivered and deployed thereby closing the durotomy. Repair of an incidental durotomy during the surgery has been shown to decrease hospital stays, reduce hospital readmissions, and lower rates of reoperation and infection. Another benefit of the implants and surgical systems disclosed herein is improved closure compared to suturing, which would result in fewer instances of CSF leaks, and ultimately fewer complications and reoperations for patients of procedures where durotomies are encountered.

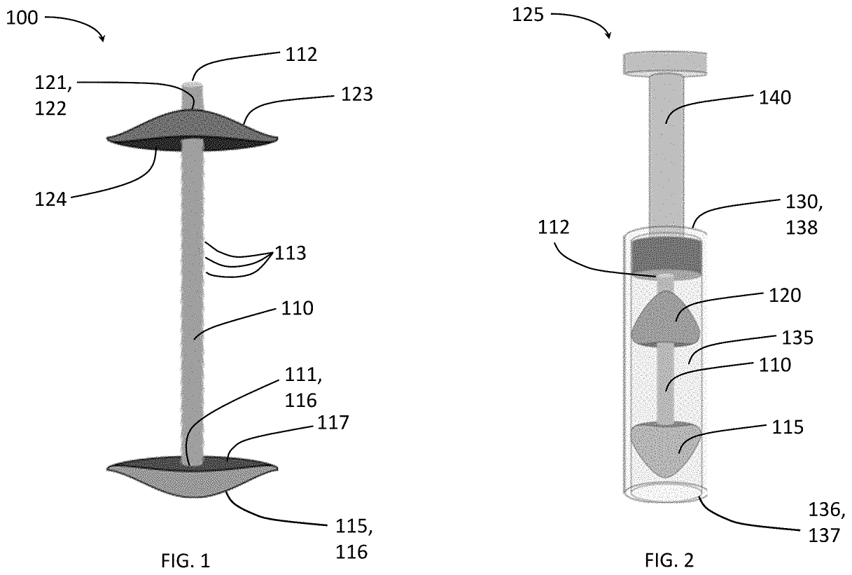

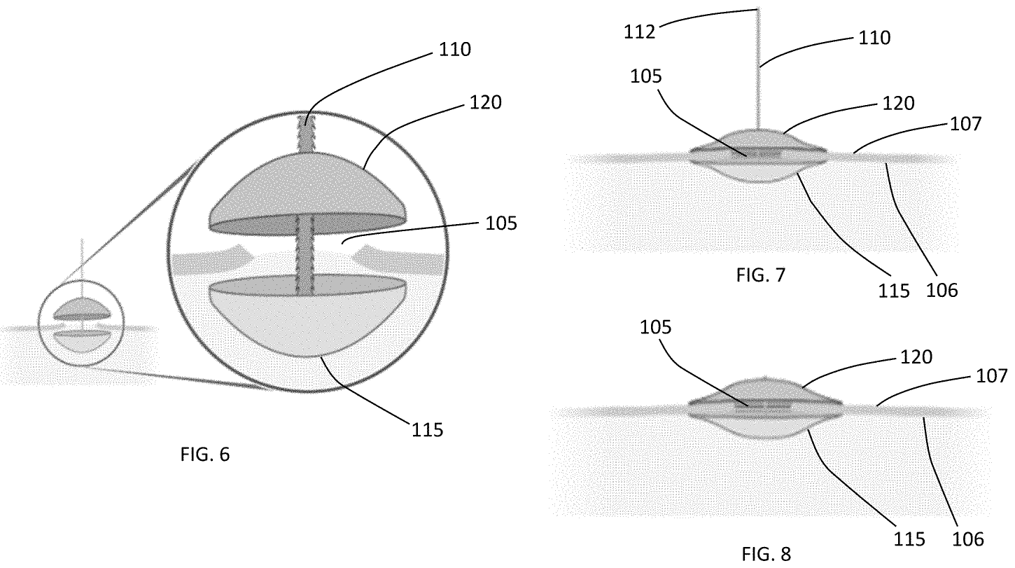

In a first aspect of the disclosure, shown in FIGS. 1-9, an implant 100 for closing a tissue opening 105 includes a barbed suture 110 having a first end 111 and a second end 112. The implant 100 also includes a first disc 115 statically coupled to the first end 111 of the barbed suture 110 at a center 116 of the first disc 115. The implant further includes a second disc 120 having a through-hole 121 arranged in a center 122 of the second disc 120, such that the second disc 120 is moveably coupled to the barbed suture 110 via the through-hole 121.

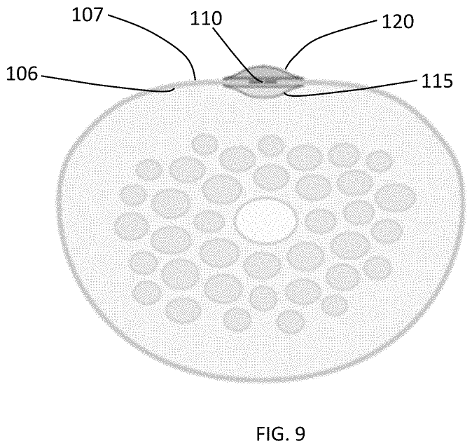

In one implementation, the barbed suture has a plurality of barbs 113 arranged unidirectionally such that the second disc 120 is configured to be advanced in a single direction toward the first disc 115. The technical effect of the uni-directional barbs 113 is to permit advancement of the second disc 120 along the barbed suture 110 toward the first disc 115 but to restrict movement of the second disc 120 toward the second end 112 of the barbed suture 110. The plurality of barbs 113 are preferably compactly spaced to minimize or eliminate play or movement of the discs 115, 120 once they are in a locked condition (FIGS. 8-9) and to promote snug facial engagement between the first disc 115 and the second disc 120 with the tissue surrounding the tissue opening 105. Once the discs 115, 120 are in the locked condition a substantially sealed chamber is formed around the tissue opening 105.

In one implementation, the first disc 115 and the second disc 120 have a delivery condition, shown in FIG. 2, and a deployment condition, shown in FIG. 1. In a further implementation, the first disc 115 and the second disc 120 are collapsed or compressed toward a central axis of each respective disc in the delivery condition (FIG. 2), and the first disc 115 and the second disc 120 expand radially outward away from a central axis of each respective disc in the deployment condition (FIG. 1).

In one optional implementation, the first disc 115 and the second disc 120 have the same diameter in the deployment condition. In a further optional implementation, the diameter of the first disc 115 and the second disc 120 is 0.5 mm to 1 mm larger than a widest point of the tissue opening 105.

In one optional implementation, the first disc 115 and the second disc 120 each have a convex face 116, 123 and a concave face 117, 124. In a further implementation, the convex face 116 of the first disc 115 is arranged facing the first end 111 of the barbed suture 110 and the concave face 117 of the first disc 115 is arranged facing the second disc 120. In addition, the convex face 123 of the second disc 120 is arranged facing the second end 112 of the barbed suture 110 and the concave face 124 of the second disc 120 is arranged facing the first disc 115. In other words, the first disc 115 and the second disc 120 may be substantially hemispherical.

In one optional implementation, the first disc 115, the second disc 120, and the barbed suture 110 are biodegradable. Additionally, materials can be selected for discs herein based on a particular application. For example, it may be preferable for certain procedures to use permanent materials, while in other cases, it may be preferable to use a biodegradable material. In another implementation, the first disc 115 and the second disc 120 are made of a biogel, a polymer, or a microplastic, as some examples. In optional implementations, the first disc 115 and the second disc 120 are formed of a malleable material.

In another optional implementation, the first disc 115 and the second disc 120 are self-expanding such that the first disc 1125 and the second disc 120 are outwardly biased towards the deployment condition (FIG. 1).

In one implementation, the second disc 120 is spaced apart from the first disc 115 along the barbed suture 110 to permit deployment of the first disc 115 on a first side 106 of the tissue opening 105 and deployment of the second disc 120 on the second side 107 of the tissue opening 105.

In a second aspect of the disclosure, shown in FIGS. 2-5, a surgical system 125 for closing a tissue opening 105 includes the implant 100 according to the first aspect of the disclosure. The surgical system 125 also includes a delivery device 130 having a lumen 135 with a first end 136 having a delivery opening 137, a second end 138, an inner diameter, and an outer diameter. The implant 100 is disposed in the lumen 135 of the delivery device 130 such that the first disc 115 is arranged adjacent to the delivery opening 137 of the lumen 135 and the second disc 120 is arranged in the lumen 135 between the first disc 115 and the second end 138 of the lumen 135. The second end 138 of the lumen 135 is coupled to a forcing imparting guide 140.

In one implementation, ss the discs are malleable, the discs 115, 120 will be folded around their concavities into a conical shape and stored in a suitable delivery syringe, the syringe having a diameter less than that of the unfolded discs 115, 120.

In one optional implementation, when the first disc 115 and the second disc 120 are in a deployment condition (FIG. 1), a diameter of each of the first disc 115 and the second disc 120 is larger than the inner diameter of the lumen 135 of the delivery device 130.

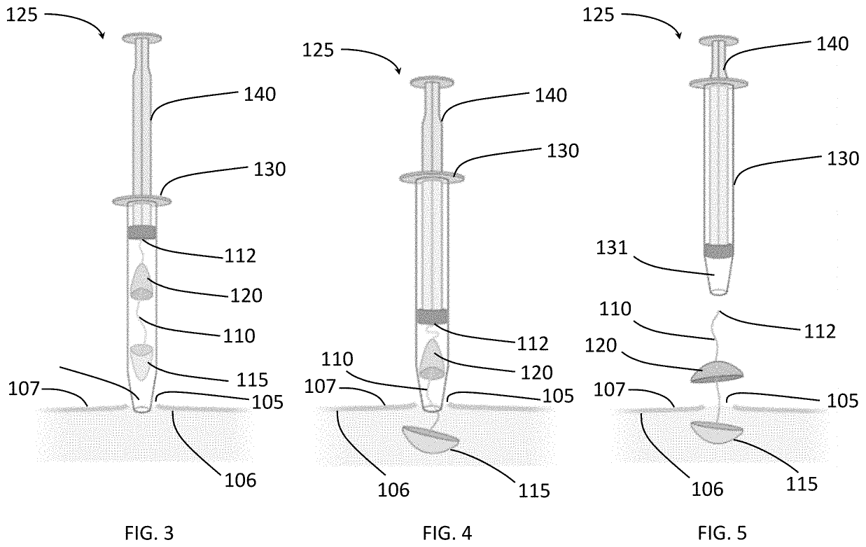

In one optional implementation, shown in FIG. 2, the inner diameter of the lumen 135 is consistent along a portion of the lumen 135 in which the implant 100 is arranged, and the inner diameter of the lumen 135 and the diameter of the deployment opening 137 are the same. In an alternative implementation, shown in FIGS. 3-5, the first end 136 of the lumen 135 is tapered. The technical effect of the tapered first end 136 is to permit easier advancement of the delivery device 130 into the tissue opening 105.

In one optional implementation, the delivery device 130 is a syringe (FIGS. 2-5), a catheter, or a cannula and the forcing imparting guide 140 is a plunger, guidewire, or trocar, respectively.

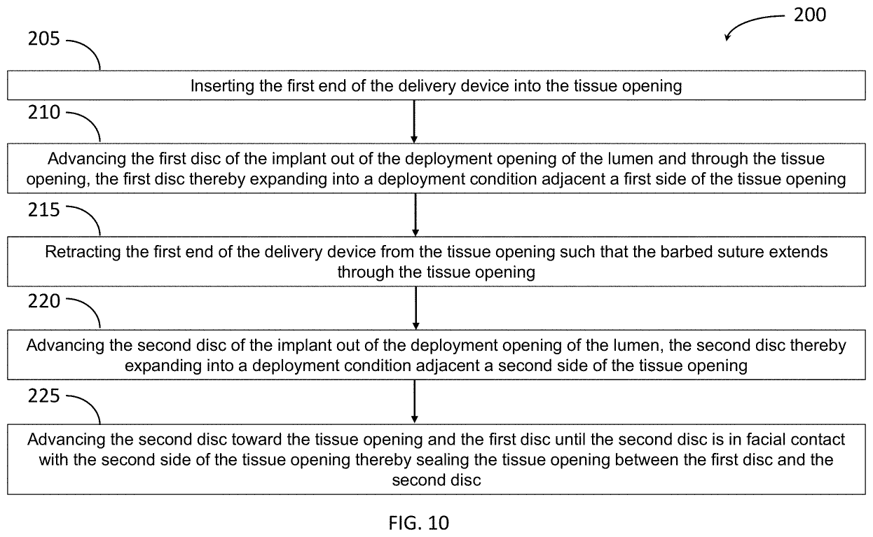

Referring now to FIG. 10, a method 200 is illustrated for closing a tissue opening 105 during a surgical procedure using the surgical system 125 according to the second aspect of the disclosure. Method 200 includes, at block 205, inserting the first end 131 of the delivery device 130 into the tissue opening 105 (FIG. 3). In one optional implementation, the first end 131 of the delivery device 130 is inserted into a durotomy. Then, at block 210, the first disc 115 of the implant 100 is advanced out of the delivery opening 137 of the lumen 135 and through the tissue opening 105, the first disc 115 thereby expanding into a deployment condition adjacent a first side 106 of the tissue opening 105 (FIG. 4). Next, at block 215, the first end 131 of the delivery device 130 is retracted from the tissue opening 105 such that the barbed suture 110 extends through the tissue opening 105. Then, at block 220, the second disc 120 of the implant 100 is advanced out of the delivery opening 137 of the lumen 135, the second disc 120 thereby expanding into a deployment condition (FIG. 5) adjacent a second side of the tissue opening. And, at block 225, the second disc 120 is advanced toward the tissue opening 105 and the first disc 115 until the second disc 120 is in facial contact with the second side 107 of the tissue opening 105 thereby sealing the tissue opening 105 between the first disc 115 and the second disc 120.

In one optional implementation, the first end 131 of the delivery device 130 is inserted into a durotomy where the first disc 115 is advanced out of the delivery device 130 into the thecal sac. Then the delivery device 130 is removed from the durotomy and the second disc 120 and second end 112 of the barbed suture 110 are advanced out of the delivery device 130 outside the thecal sac. The barbed suture 110 can be pulled taut by the surgeon so that the first disc 115 is pulled tight against the first side 106 of the tissue thereby covering the durotomy in its entirety. Then, while the barbed suture 110 is still under tension, the surgeon slides the second disc 120 down the barbed suture 110 onto the second side 107 of the tissue bringing the first disc 115 and the second disc 120 together and thereby sandwiching the dura in between them and sealing the durotomy closed. The excess barbed suture 110 may then be cut from an outside top surface of the second disc 120.

The implant 100, surgical system 125, and method 200 in accordance with the principles herein represent an intraoperative mode of durotomy repair that would both reduce the amount of time it takes to repair the durotomy and expand the proportion of durotomies that can be primarily closed. Additionally, this the implant 100 and surgical systems 125 could be used in conjunction with current methods of secondary closure, as the implant 100 can be used to fasten a dural patch to the durotomy, and dural sealant can be used over the exterior surface of the implant 100 after deployment.

In operation, when repairing the dura mater, there will be an egress of fluid displaced out of the subarachnoid space by the implant 100 due to the perforation or tissue opening 105 that would prevent a build up of pressure at the treatment location. Still further, since the first disc 115 is pulled tightly against the first surface 106 of the tissue or the dura, the first disc 115 will not reside against neural elements. In other words, there is no pressure applied to neural elements. Once the implant 100 is deployed and the tissue opening 105 is sealed, further outflux of CSF would be prevented. Still further, a majority of spinal CSF leaks or dural tears occur in the lumbar and sacral spine. As such, the implant would typically be delivered to the L2-S2 vertebral levels, where the spinal cord is not present.

In one optional implementation, the method 200 further includes removing a portion of the barbed suture 110 from a convex side 123 of the second disc 120 after the tissue opening 105 is sealed (FIGS. 8-9). During this step, the barbed suture 110 may be pulled taut and clipped near the convex 123 face of the second disc 120.

In one optional implementation, shown in FIGS. 2-5, the delivery device 130 is a syringe, and the first disc 115 and the second disc 120 are advanced out of the lumen 135 via a plunger 140.

In one optional implementation, the second disc is manually advanced along the barbed suture 110 via a force applied to the convex face 123 of the second disc 120. For example, forceps may be used to push the second disc 120 along the barbed suture 110 or a surgeon may use several fingers to push the disc 120 along the barbed suture 100 to seal the tissue opening 105 between the two discs 115, 120.

The description of different advantageous arrangements has been presented for purposes of illustration and description and is not intended to be exhaustive or limited to the examples in the form disclosed. Many modifications and variations will be apparent to those of ordinary skill in the art. Further, different advantageous examples may describe different advantages as compared to other advantageous examples. The example or examples selected are chosen and described in order to best explain the principles of the examples, the practical application, and to enable others of ordinary skill in the art to understand the disclosure for various examples with various modifications as are suited to the particular use contemplated.

1. An implant for closing a tissue opening, the implant comprising:
a barbed suture having a first end and a second end;
a first disc statically coupled to the first end of the barbed suture at a center of the first disc; and
a second disc having a through-hole arranged in a center of the second disc, wherein the second disc is moveably coupled to the barbed suture via the through-hole.

2. The implant according to claim 1, wherein the barbed suture has a plurality of barbs arranged unidirectionally such that the second disc is configured to be advanced in a single direction toward the first disc.

3. The implant according to claim 1, wherein the first disc and the second disc have a delivery condition and a deployment condition.

4. The implant according to claim 3, wherein the first disc and the second disc are collapsed or compressed toward a central axis of each respective disc in the delivery condition and wherein the first disc and the second disc expand radially outward away from a central axis of each respective disc in the deployment condition.

5. The implant according to claim 1, wherein the first disc and the second disc have the same diameter in the deployment condition.

6. The implant according to claim 5, wherein the diameter of the first disc and the second disc is 0.5 mm to 1 mm larger than a widest point of the tissue opening.

7. The implant according to claim 1, wherein the first disc and the second disc each have a convex face and a concave face.

8. The implant according to claim 7, wherein the convex face of the first disc is arranged facing the first end of the barbed suture and the concave face of the first disc is arranged facing the second disc, and wherein the convex face of the second disc is arranged facing the second end of the barbed suture and the concave face of the second disc is arranged facing the first disc.

9. The implant according to claim 1, wherein the first disc, the second disc, and the barbed suture are biodegradable.

10. The implant according to claim 1, wherein the first disc and the second disc are self-expanding such that the first disc and the second disc are outwardly biased towards the deployment condition.

11. The implant according to claim 1, wherein the second disc is spaced apart from the first disc along the barbed suture to permit deployment of the first disc on a first side of the tissue opening and deployment of the second disc on the second side of the tissue opening.

12. A surgical system for closing a tissue opening comprising:
the implant according to claim 1; and
a delivery device having a lumen with a first end having a delivery opening, a second end, an inner diameter, and an outer diameter, wherein the implant is disposed in the lumen of the delivery device such that the first disc is arranged adjacent to the delivery opening of the lumen and the second disc is arranged in the lumen between the first disc and the second end of the lumen, and wherein the second end of the lumen is coupled to a force-imparting guide.

13. The surgical system of claim 12, wherein, when the first disc and the second disc are in a deployment condition, a diameter of each of the first disc and the second disc is larger than the inner diameter of the lumen of the delivery device.

14. The surgical system of claim 12, wherein the inner diameter of the lumen is consistent along a portion of the lumen in which the implant is arranged, and the inner diameter of the lumen and the diameter of the deployment opening are the same.

15. The surgical system of claim 12, wherein the first end of the lumen is tapered.

16. The surgical system of claim 12, wherein the delivery device comprises a syringe, a catheter, or a cannula.

17. A method of closing a tissue opening during a surgical procedure using the surgical system according to claim 12, the method comprising:
inserting the first end of the delivery device into the tissue opening;
advancing the first disc of the implant out of the delivery opening of the lumen and through the tissue opening, the first disc thereby expanding into a deployment condition adjacent a first side of the tissue opening;
retracting the first end of the delivery device from the tissue opening such that the barbed suture extends through the tissue opening;
advancing the second disc of the implant out of the delivery opening of the lumen, the second disc thereby expanding into a deployment condition adjacent a second side of the tissue opening; and
advancing the second disc toward the tissue opening and the first disc until the second disc is in facial contact with the second side of the tissue opening thereby sealing the tissue opening between the first disc and the second disc.

18. The method of claim 17, further comprising:
removing a portion of the barbed suture from a convex side of the second disc after the tissue opening is sealed.

19. The method of claim 17, wherein the delivery device comprises a syringe, and wherein the first disc and the second disc are advanced out of the lumen via a plunger.

20. The method of claim 17, wherein the second disc is manually advanced along the barbed suture via a force applied to the convex face of the second disc.
